(19) United States
(12) Patent Application Publication
(10) Pub. No.: US 2002/0022664 A1
(43) Pub. Date: Feb. 21, 2002

(54) Method for treating aberrant fibrotic tissue manifestations with topical calcium channel blocker preparations and improved composition for such treatment and improved method for the manufacture thereof

(76) Inventor: W. Jerry Easterling, San Antonio, TX (US)

Correspondence Address: DAVID G. HENRY, 900 Washington Ave., P.O. Box 1470, Waco, TX, 76703-1470, US

(21) Appl. No.: 09/839,604

(22) Filed: Apr. 20, 2001

Related U.S. Application Data
(63) Continuation-in-part of application No. 09/514,796, filed on Feb. 28, 2000, now abandoned.
Application No. 09/514,796 is a continuation-in-part of application No. 09/128,103, filed on Aug. 3, 1998, now Pat. No. 6,031,005.

(51) Int. Cl. A61K 31/135

(52) U.S. Cl. 514/654

(57) ABSTRACT

The invention is of an improved to topical medicament and associated methodology for use thereof, through the use of which aberrant fibrotic tissue manifestations involving scarring or sub-dermal plaque formations or accumulations may be effectively, cost effectively, and painlessly treated. One or more calcium channel blocker agents serve as the primary active ingredient of the present compositions and transdermal penetration agents or carriers are included to facilitate topical delivery of the active ingredient(s) to the intended, sub-dermal treatment site.

### CITATION TO PRIOR APPLICATION

This is a continuation-in-part with respect to U.S. patent application, Ser. No. 09/514,796 filed Feb. 28, 2000 which was a continuation-in-part of U.S. patent application Ser. No. 09/128,103 (now U.S. Pat. No. 6,031,005), from which application and its parent application priority is here claimed under 35 U.S.C. §120.

### BACKGROUND OF THE INVENTION

1. Field of The Invention

Applicant's invention relates to medicaments and treatment procedures relating to Peyronie's disease and related connective tissue maladies.

2. Background Information

In U.S. Pat. No. 6,031,005 (and subsequently filed continuation-in-part applications in relation thereto, which CIPs have not issued at the time of this filing), the present inventor has provided new and unobvious treatment regimens for a variety of fibrotic conditions through the use of topically applied calcium channel blocker-based preparations. The specification of U.S. Pat. No. 6,031,005 (“the '005 patent”) is incorporated herein by reference, as if set forth herein verbatim.

The preparations and associated methods for the topical application of calcium channel blocker preparations as taught in the '005 patent have proven remarkably effective in treating, not only the conditions described in the'005 patent (notably Peyronie's disease, Dupuytren's Hand Contracture, Ledderhose Fibrosis and scarring), but also in treating hemangiomas, “spider veins” and “cellulite.” However, the thus far preferred embodiment and best known mode of the topical calcium channel blocker preparations of the '005 patent's invention (the verapamil-based composition as taught in the '005 patent), both in 10% and 15% strengths have demonstrated instability as evidenced by the formation of crystals.

The time over which crystals have formed in topical verapamil formulations has varied dramatically, from as little as 30 days to as long as 90 days after formulation. Some batches of topical verapamil preparations have deteriorated, while others do not appear to have done so. These inconsistencies suggested to the present inventor that the chemical reactions involved were intiated by more than one material cause.

Prior to deterioration, the topical verapamil preparations as taught by the '005 patent performed beyond anyone's reasonable expectations in treating various aberrant fibrotic conditions. However, once deterioration reaches a detectable level, difficulty in accurately dispensing the preparations comes into play and at least calls into question the level of efficacy to be expected from use of the preparations because of the apparent chemical changes having occurred (with possible reduction in active ingredients).

Therefore, it became imperative, at least in the view of the present inventor, that a new formulation be found which would alleviate the deterioration problem with the topical verapamil formulations, particularly if the formulations were to be distributed as an FDA-approved, off-the-shelf pharmaceutical product, rather than a formulated-upon-demand (by prescription) medication. Of course, such new formulation should not be made at the expense of efficacy.

### SUMMARY OF THE INVENTION

It is an object of the present invention to provide an improved medicament useful in the treatment of aberrant fibrotic tissue manifestations which are exemplified by sub-dermal plaque or scar tissue formation or accumulations, such as Peyronie's disease, Dupuytren's contracture, Ledderhose Fibrosis, scars, hemangiomas, and lipoederma (“cellulite”).

It is another object of the present invention to provide an improved medicament useful in the treatment of aberrant fibrotic tissue manifestations which are exemplified by sub-dermal plaque or scar tissue formation or accumulations, such as Peyronie's disease, Dupuytren's contracture, Ledderhose Fibrosis, scars, hemangiomas, and lipoederma (“cellulite”), which medicament is more stable than prior medicaments of the same nature and by the same inventor.

It is an object of the present invention to provide an improved medicament useful in the treatment of aberrant fibrotic tissue manifestations which are exemplified by sub-dermal plaque or scar tissue formation or accumulations, such as Peyronie's disease, Dupuytren's contracture, Ledderhose Fibrosis, scars, hemangiomas, and lipoederma (“cellulite”), which medicament is applications when topically applied and thereby obviates the need for invasive treatment regimens for such conditions.

It is another object of the present invention to provide and improved method for treating aberrant fibrotic tissue manifestations which are exemplified by sub-dermal plaque or scar tissue formation or accumulations, such as Peyronie's disease, Dupuytren's contracture, Ledderhose Fibrosis, scars, hemangiomas, and lipoederma (“cellulite”).

It is another object of the present invention to provide and improved method and medicament for treating severe bums whereby the injury is managed such that dermal rupturing and subsequent scarring are mitigated.

It is another object of the present invention to provide and improved method for treating aberrant fibrotic tissue manifestations which are exemplified by sub-dermal plaque or scar tissue formation or accumulations, such as Peyronie's disease, Dupuytren's contracture, Ledderhose Fibrosis, scars, hemangiomas, and lipoederma (“cellulite”), which method involves the use of an improved medicament which is more stable than prior medicaments of the same nature and by the same inventor.

It is another object of the present invention to provide and improved method for preparing a topical calcium channel blocker preparation for use in treating aberrant fibrotic tissue manifestations which are exemplified by sub-dermal plaque or scar tissue formation or accumulations, such as Peyronie's disease, Dupuytren's contracture, Ledderhose Fibrosis, scars, hemangiomas, and lipoederma (“cellulite”), which method involves the addition of heretofore omitted elements, the addition of which yields a substantially more shelf stable product than the prior medicaments of the same nature and by the same inventor.

In satisfaction of these and related objectives, Applicant's present invention provides an improved topical medicament and associated methodologies for preparation and use thereof, through the use of which medicament, through topical application, aberrant fibrotic tissue manifestations exemplified by sub-dermal plaque or scar tissue formation or accumulations may be treated, such conditions including, without limitation, Peyronie's disease, Dupuytren's contracture, Ledderhose Fibrosis, existing scars, hemangiomas, and lipoederma (“cellulite”). The present medicament also shows great promise in treating severe burns.

The invention, although exemplified by specific embodiments which are based upon, or rely on the use of specific calcium channel blockers, is not limited to such species. Rather, observations by the present inventor indicate that when coupled with a suitable carrier for transdermal delivery, all thus-far-evaluated calcium channel blocker-based preparations (regardless of the species of calcium channel blocker used) effect reduction of aberrant fibrotic tissue manifestations. Therefore, the true scope of the invention encompasses preparations and methods of use facilitating or involving the use of transdermal application of calcium channel blockers in the treatment of aberrant fibrotic tissue manifestations which involve sub-dermal plaque or scar tissue accumulations (Peyronie's disease, Dupuytren's contracture, and Ledderhose Fibrosis, existing scarring “spider veins” and cellulite, for example). The medicaments of the present invention also useful in managing and mitigating the long-term damage caused by burn wounds.

The medicament of the present invention is an improved, quite shelf-stable topical gel which, like its predecessor as taught in the '005 patent, repeatably and predictably effects complete, or near complete reversal of perceptible aberrant fibrotic tissue manifestations. In the case of Peyronie's disease, symptoms in the majority of users, and in all cases, effects a substantial reduction of such symptoms to a substantially greater degree and substantially higher incidence than previously experienced by patient populations over-all, and in individual instances wherein patients had previously attempted conventional treatment regimens.

### DETAILED DESCRIPTION OF THE PREFERRED EMBODIMENT

In the preferred embodiment of the present medicament, and in the medicament upon which the associated method are based, the primary active ingredient is Verapamil Hydrochloride, USP (a diphenylalkylamine). However, it should be understood that other calcium channel blockers (topically applied in a similar composition) provide similar relief. With certain patients, combinations of channel blocker agents seem to have an even greater efficacy than the single, Verapamil agent. Other such calcium channel blockers include benzothiazepines (Diltiazem, for example), dihydropyridines (Amlodipine, Felodipine, Isradipine, Nicardipine, Nifedipine, Nimodipine, or Nisoldipine), and the fast sodium inward channel inhibitor—Bepridil.

### I. Basis of Formulation Changes

In evaluating the deterioration problems with the prior embodiments of the present inventor's medicaments, the present inventor may get the following observations and/or came to certain conclusions:

1. Air is being entrained into the materials at all stages of formulation.

2. The ethoxydiglycol reagent is reacting with the air and forming byproducts including but not limited to aldehydes, peroxides, and free radicals which cause drug crystallization and subsequent loss of therapeutic potency. Additionally, these byproducts can cause skin irritation.

3. Verapamil is a chemical derivative of papaverine. Papaverine, in the presence of heavy metals, will deteriorate rapidly. The verapamil formulations may be affected by the presence of heavy metal ions that originate from the mixing containers or equipment.

Based upon these conclusions, the present inventor made the following basic changes to his prior formulations and preparation steps:

1. Butylated hydroxytoluene (BHT), NF. BHT is added, and serves as an antioxidant to counteract any reaction with entrained air.

2. Nitrogen, NF, is used to purge all containers during chemical addition and mixing. Every ointment tube is purged just prior to filling and sealing. The nitrogen serves as a replacement for entrained air and is non-reactive with the components.

3. A “non-reactive” glaminate ointment tube is used so that no reaction occurs with the ointment tube.

4. Edetate disodium, USP is added to the gel formulation and serves as a chelating agent to bind any heavy metal ions and prevent reaction of same.

5. Propylene glycol, USP has been added as an additional drug solvent and skin absorption enhancer.

The result of making the preceding changes to the prior gel formulations is a gel which is stable over periods of many months, even after undergoing formal, rigorous stability studies by an independent pharmaceutical laboratory. Patient evaluations indicate that the change in formulation has in no way negatively affected efficacy and, and fact, appears to have somewhat enhanced such efficacy.

### II. Preparation

The now-preferred Verapamil-based gels of the present invention (in exemplary 10% and 15% percent strengths) may be prepared according to the following disclosure and protocol, with variations appropriate to a desired scale of production as will be apparent to persons skilled in the production of pharmaceutical preparations:

A. Constituents of Preferred Embodiment of Topical Verapamil Gel 10% and 15%

| Ingredients | 10% (% W/W) | 15% (% W/W) |
|---|---|---|
| Verapamil | 10.0 | 15.0 |
| Ethoxydiglycol | 14.0 | 19.5 |
| Propylene Glycol | 0.5 | 0.5 |
| Butylated Hydroxy Toluene (BHT) | 0.1 | 0.1 |
| Lecithin Soya Granular | 13.1 | 13.1 |
| Isopropyl Myristate | 13.1 | 13.1 |
| Sorbic Acid | 0.09 | 0.09 |
| Pluronic F127 | 9.8 | 11.6 |
| Potassium Sorbate | 0.15 | 0.12 |
| Disodium Edetate | 0.01 | 0.01 |
| Purified Water | 39.15 | 26.88 |

B. Topical Verapamil 15% (To Make 3000 Gm)

| Ingredients | Quantity | |
|---|---|---|
| Verapamil HCI USP | 450.00 | Gm |
| Ethoxydiglycol Reagent | 585.0 | Gm |
| Lecithin/Isopropyl Myristate Solution | 790.0 | Gm |
| Butylated Hydroxytolune NF (BHT) | 3.0 | Gm |
| Edetate Disodium USP | 0.30 | Gm |
| Propylene Glycol USP | 15.0 | Gm |
| Pluronic Gel 30% | 1,156.7 | Gm |

Instructions: Dissolve verapamil in ethoxydiglycol and propylene glycol with the aid of heat (90-100 degrees C.). Stir during this dissolving step. When the solution is clear, weigh to ascertain the amount of evaporation. Add the amount lost to evaporation back as ethoxydiglycol. Immediately add the lecithin/isopropyl myristate and BHT and stir well. Weigh the PLO 30% into a plastic container, add edetate disodium and stir gently to dissolve edetate disodium. Avoid foaming with stirring. Gently add the verapamil phase to the PLO phase, avoiding the incorporation of air. Stir for 10 minutes using a 3 inch mixing blade at 31OO rpm. Dispense in 3O Gm glaminate ointment tubes.

C. Topical Verapamil 10% (To Make 3000 Gm)

| Ingredients | Quantity | |
|---|---|---|
| Verapamil HCI USP | 300.00 | Gm |
| Ethoxydiglycol Reagent | 420.0 | Gm |
| Lecithin/Isopropyl Myristate Solution | 790.0 | Gm |
| Butylated Hydroxytolune NF (BHT) | 3.0 | Gm |
| Edetate Disodium USP | 0.30 | Gm |
| Propylene Glycol USP | 15.0 | Gm |
| Pluronic Gel 30% | 1,471.7 | Gm |

Instructions: Dissolve verapamil in ethoxydiglycol and propylene glycol with the aid of heat (90-100 degrees C.). Stir during this dissolving step. When the solution is clear, weigh to ascertain the amount of evaporation. Add the amount lost to evaporation back as ethoxydiglycol. Immediately add the lecithin/isopropyl myristate and BHT and stir well. Weigh the PLO 30% into a plastic container, add edetate disodium and stir gently to dissolve edetate disodium. Avoid foaming with stirring. Gently add the verapamil phase to the PLO phase, avoiding the incorporation of air. Stir for 5 minutes using a 3 inch mixing blade at 31OO rpm. Dispense in 3O Gm glaminate ointment tubes.

D. Pluronic Gel 20% (To Make 3000 Gm)

| Ingredients | Quantity | |
|---|---|---|
| Pluronic F127 NF (Poloxamer 407) | 600.00 | Gm |
| Potassium Sorbate NF | 9.00 | Gm |
| Water (Sterile for Irrigation) qs to | 3,000.00 | Gm |

Directions: Prepare a pluronic gel by combining the potassium sorbate and pluronic F 127 and bringing to a total weight of 3,000 Gm. with cold (refrigerated) sterile water. Make sure that all the granules are wet, and place in a refrigerator. Mixture will form a clear solution over 24-48 hours.

Alternate Procedure: The above mixture can be uniformly mixed with a mixing blade. It will take on the appearance of beaten egg whites. When placed in the refrigerator it will form a clear solution much faster, usually overnight.

The above solution will solidify into a clear gel at room temperature.

E. Pluronic Gel 30% (To Make 2000 Gm)

| Ingredients | Quantity | |
|---|---|---|
| Pluronic F 127 NF (Poloxamer 407) | 600.00 | Gm |
| Potassium Sorbate NF | 6.00 | Gm |
| Water (Sterile for Irrigation) qs to | 2,000.00 | Gm |

Instructions: Prepare a pluronic gel by combining the potassium sorbate and pluronic F 1 27 and bringing to a total weight of 2,000 Gm. with cold (refrigerated) sterile water. Make sure that all the granules are wet, and place in a refrigerator. Mixture will form a clear solution over 24-48 hours.

Alternate Procedure: The above mixture can be uniformly mixed with a mixing blade. It will take on the appearance of beaten egg whites. When placed in the refrigerator it will form a clear solution much faster, usually overnight. The above solution will solidify into a clear gel at room temperature.

F. Lecithin/Isopropyl Myristate Solution (To Make 3000 Gm)

| Ingredients | Quantity | |
|---|---|---|
| Lecithin Soya Granular | 1,494.0 | Gm |
| Isopropyl Myristate NF | 1,494.0 | Gm |
| Sorbic Acid NF Powder | 9.90 | Gm |

Instructions: Disperse lecithin and sorbic acid in isopropyl myristate. Allow to stand at room temperature until a liquid of syrup consistency forms. Stir well and store in a light protected container.

### III. Use of Preparations

The choice of strengths of the topical verpamil gels taught above will depend on the experience of the clinician. Ordinarily, a patent with a fibrotic condition will be started with the lower dosage preparation, and only if the patient fails to respond, or responds more slowing than reasonably would be expected, would the patient be changed to the higher dosage form.

In any event, use of all topical calcium channel blocker preparations of the present inventor's work involves simply applying a thin coating of the gels to an affected area or bodily structure, usually once daily. Clinicians will prescribe certain volumetric dosages, which dosages can be metered by any number of conventional metering means (syringes, dosimeters, blister packs, single-dose tubes, etc.)

In the case of Peyronie's disease, the patient should apply the medication by starting at the point where the plaque is heaviest or where the curvature begins and work out until the entire penile shaft has been covered with medication. In the case of Dupuytren's Contracture, the patient should coat the cords and immediately surrounding areas with the prescribed dosage of the gels, with the same basic approach being applicable to treating Ledderhose fibrosis of the feet. In the case of scars and hemangiomas, the gels are applied to the visible deformation and the immediately surrounding areas. In the case of “cellulite”, the gels are applied generally to the affected area.

Although the invention has been described with reference to specific embodiments, particularly with respect to the particular active ingredient of the present medicament, this description is not meant to be construed in a limited sense, in particular to limit the scope of the appended claims to cover only those medicaments and associated modalities of treatment which include Verapamil as the calcium channel blocker, the function of which in the area of plaque appears to lie at the heart of the efficacy of the present medicament. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention.

I claim:

1. A medicament for use in the treatment of aberrant fibrotic tissue manifestations comprising:
a carrier host agent for facilitating transdermal application of a calcium channel blocker agent to an affected bodily structure;
a calcium channel blocker agent suspended in said carrier host agent; and
an antioxidant agent suspended in said carrier host agent for preventing the oxidation of active ingredients of said medicament.

2. The medicament of claim 1 wherein said calcium channel blocker agent is verapamil.

3. The medicament of claim 1 wherein said medicament comprises:
verapamil;
a lecithin/isopropyl myristate solution;
butylated hydroxy toluene;
pluronic F127; and
water.

4. The medicament of claim 3 further comprising:
Edetate disodium.

5. The medicament of claim 3 further comprising:
Propylene glycol.

6. The medicament of claim 4 further comprising:
Propylene glycol.

7. A method for treating aberrant fibrotic tissue manifestations comprising the steps of:
selecting a medicament comprising:
carrier host agent for facilitating transdermal application of a calcium channel blocker agent to a bodily structure having an aberrant fibrotic tissue manifestation;
a calcium channel blocker agent suspended in said carrier host agent; and
an antioxidant agent suspended in said carrier host agent to four preventing oxidation of constituents of said medicament;
periodically, topically applying a therapeutic dosage of said medicament to a bodily structure which exhibits symptoms of a connective or elastic tissue disease.

8. The method of claim 3 wherein said calcium channel blocker agent is verapamil.

9. The method of claim 7 wherein said calcium channel blocker agent is selected from the chemical groups consisting of diphenylalkylamine, benzothiazepines, or dihydropryridines.
